(19) United States
(12) Patent Application Publication
(10) Pub. No.: US 2014/0106329 A1
(43) Pub. Date: Apr. 17, 2014

(54) METHOD OF PRODUCING ORGAN MODEL, MOLD FOR PRODUCING ORGAN MODEL, AND ORGAN MODEL

(71) Applicant: JMC CORPORATION, Yokohama-shi (JP)

(72) Inventors: Daichi WATANABE, Yokohama (JP); Makoto Inada, Yokohama (JP)

(73) Assignee: JMC CORPORATION, Yokohama-shi (JP)

(21) Appl. No.: 14/141,155

(22) Filed: Dec. 26, 2013

Related U.S. Application Data
(63) Continuation of application No. PCT/JP2013/068822, filed on Jul. 10, 2013.

(30) Foreign Application Priority Data
Jul. 13, 2012 (JP) 2012-157201

(51) Int. Cl. G09B 23/30; A61B 10/00

(52) U.S. Cl. CPC G09B 23/30; A61B 10/00
USPC 434/272; 264/219; 425/2

(57) ABSTRACT

The present invention provides a method of producing an organ model comprising; an outer-shape body forming step in which an outer-shape body 120 having regions 16, 17 to be a hollow portion and a structural wall of the organ model respectively is formed by irradiating curing light and cure the photocurable mold resin 12 and support resin 13 supporting the mold resin based on photographed data of a human organ, a mold shell forming step in which a mold shell 10 having outer and inner shell portions 12A, 12B covering outer and inner surfaces of the organ model respectively is formed by removing the support resin 13 from the outer-shape body 120, a filling step in which a space 15 between the outer and inner shell portions 12A, 12B is filled with a flexible injection molding material 20, and a removing step for removing the mold shell 10.

### TECHNICAL FIELD

The present invention relates to a method for producing an organ model (phantom) which is a stereo model of various organs existing inside a living body such as a human body, a mold for producing the organ model used in such a method, and the organ model which is produced using the method and/or the mold.

### BACKGROUND ART

Conventionally, an X-ray apparatus, a CT scanning apparatus, an ultrasonic diagnostic apparatus, or the like is used in medical sites. A doctor understands the state of a lesion from data (2-dimensional data such as a photograph and image data) obtained from such apparatuses, or performs actual operations referring to such data. On performing an actual operation on a human body, it is important to understand the target lesion not only by 2-dimensional data such as a photograph but in a 3-dimensional manner. For example, in an operation for treating a heart by a catheter, it is preferable to previously understand the specific structure of the portion to be treated (a blood vessel in which a catheter is actually inserted and a portion in which a stent is positioned) in a 3-dimensional manner.

As for an operation using a catheter, a practical training using an animal such as a pig as an object is performed as a previous step before an actual operation is performed on a human body. However, it is not sufficient since the structure of an organ of a human is different from that of an animal due to the difference of the basic biological structure between the human and the animal. Further, when such animal is used as an object for the training in which the catheter is operated, the behavior of the catheter in a heart structure of an actual human body cannot visually be understood.

Therefore, for example, in Patent Literature 1 and Patent Literature 2, an art of actually producing a stereo organ model from image data derived from a CT scanner is disclosed. Such known art is for producing a stereo organ model by the rapid prototyping (optical prototyping), in which laser light is irradiated to a photocurable resin to produce a stereo organ model based on a 2-dimensional image data obtained by a CT scanner or the like.

The above-mentioned organ model produced by such rapid prototyping has hardness extremely higher than an actual human organ, and is not flexible as the organ since the organ model is composed of a photocurable resin. Therefore, the behavior of the catheter during the operation as mentioned above is different, not to mention the difference in the feel of touch, so that such organ model is not suitable for simulating the actual operation. Further, it is difficult to produce an organ model which provides the feel of touch similar to that of the actual human organ and precisely copies the shape of the actual human organ, easily with low cost.

In Patent Literature 3, a method by which a heart model is produce by using an outer mold and a core which are formed using the rapid prototyping is disclosed. Specifically, the outer mold is produced by a master model of a heart, and 3-dimensional data of the profile of a hollow portion of the heart is produced by tomogram data of the heart. The core is produced by shifting (offsetting) the 3-dimensional data by the thickness from. The core is set in the outer mold and a soft resin material is injected in a gap between the outer mold and the core. The mold is removed and then the core is crushed and ejected to produce an organ (heart) model.

Further, the applicant proposes in Patent Literature 4, which is an earlier application, a method in which a mold for producing an organ model is formed using the rapid prototyping and an actual organ model is produced using the mold. In the method of producing the organ model, an outer-shape body and an inner-shape body of an organ are formed by the rapid prototyping. A split mold having an internal space (base mold) is produced by using the outer-shape body, and a split mold for forming a core (core mold) is produced by using the inner-shape body. Then, an actual core is formed by the core mold. The core is set in a position so as that a given space is provided between the base mold, and a flexible thermoplastic resin is injected in the space between the base mold and the core. After the thermoplastic resin is cured, the core is melted and removed to produce a flexible organ model.

### CITATION LIST

### Patent Literature

Patent Literature 1: JP H05 (1993)-11689 A
Patent Literature 2: JP H08 (1996)-18374 A
Patent Literature 3: WO 2012/001803 A1
Patent Literature 4: JP 2010-287813 A

### SUMMARY OF INVENTION

### Technical Problem

However, a human organ has a highly complicated shape (internal shape is especially complicated). Therefore, there exists a problem that in the method of producing an organ model using the outer mold and the core as mentioned above, it is difficult to determine the direction to remove the mold and the location to split the mold, thereby making it hard to produce a precise model. Further, before producing a final mold, the outer-shape body and the inner-shape body of the organ are formed as well as the base mold (outer mold) and the core by copying the outer-shape body and the inner-shape body. This makes the production process complicated, raises the product cost, and also deteriorates accuracy. Further, in the case of copying an organ model unique to a patient, there is a problem that producing such mold as mentioned above results in the rise in cost of the mold.

The present invention is made in view of the above-mentioned problem. The first object of the present invention is to provide a method of producing an organ model in which a stereo organ model formed of a material having flexibility close to that of an actual human organ can be produced with low cost and high accuracy, and an organ model produced by such a method of producing. Further, the second object of the present invention is to provide a mold for producing an organ model which enables to produce such organ model.

### Solution to Problem

In order to solve the above-mentioned objects, the present invention provides a method of producing an organ model having a hollow portion inside thereof. The method includes: an outer-shape body forming step in which an outer-shape body having a region which is to be the hollow portion and a region which is to be a structural wall of the organ model is formed by irradiating a photocurable mold resin and a photocurable support resin which supports the mold resin with curing light so as to cure the mold resin with support from the support resin based on photographed data of a human organ; a mold shell forming step in which a mold shell having an outer shell portion which covers an outer surface of the organ model and an inner shell portion which covers an inner surface of the organ model is formed by removing the support resin from the outer-shape body; a filling step in which a space between the outer shell portion and the inner shell portion of the mold shell is filled with a flexible injection molding material; and a removing step in which the mold shell filled with the injection molding material is removed.

In the method as mentioned above, firstly, based on photographed data of a human organ, curing light, for example, laser light or ultraviolet light from an ultraviolet lamp, is irradiated to a photocurable mold resin and a photocurable support resin which supports the mold resin. Thereby, the mold resin is cured with the support from the support resin to form an outer-shape body having a region which is to be a hollow portion of the organ model and a region which is to be a structural wall of the organ model. The outer portion (surface) of the outer-shape body is formed of the mold resin, and in the internal portion of the outer-shape body, the mold resin is kept in a given shape and held in air (in a floating condition) by the support resin.

By removing the support resin from the outer-shape body which is formed as mentioned above, a mold shell including an outer shell portion which covers the outer surface of the organ model and an inner shell portion which covers the inner surface of the organ model is formed with the mold resin. In this case, the inner surface side of the outer shell portion matches the shape of the surface of the organ model, and the outer surface side of the inner shell portion matches the shape of the surface facing the hollow portion of the organ model.

Further, the space between the outer shell portion and the inner shell portion of the mold shell formed as mentioned above is filled with a flexible injection molding material. The injection molding material itself forms the organ model. When the injection molding material is cured, the mold shell is removed (destroyed) so that the organ model which precisely copies the photographed human organ can be obtained.

The above-mentioned outer-shape body precisely copies the outer shape and the inner shape of the human organ, based on the photographed data of the human organ, by the rapid prototyping technique. By removing the support resin, the mold (mold shell) for producing the organ model from the mold resin is produced. The mold (mold shell) is thus produced as a precise copy from the photographed data of the human organ. By filling the mold with a flexible injection molding material, particularly, an injection molding material having hardness close to that of the actual human organ, an organ model of which condition is close to the human organ can be obtained.

### Advantageous Effects of Invention

According to the present invention, a stereo organ model formed of a material having flexibility close to that of an actual human organ can be produced with low cost and high accuracy.

### BRIEF DESCRIPTION OF DRAWINGS

FIG. 1 is a figure illustrating a general schematic shape of an organ model (heart model) produced based on the method according to the present invention.

FIG. 2 is a figure illustrating a mold (mold shell) for producing the heart model illustrated in FIG. 1 in which a right atrium side is illustrated in a cross section.

FIG. 3A is a figure illustrating the first step of a schematic process of producing a heart model by the rapid prototyping technique.

FIG. 3B is a figure illustrating the second step of the schematic process of producing a heart model by the rapid prototyping.

FIG. 3C is a figure illustrating the third step of the schematic process of producing a heart model by the rapid prototyping technique.

FIG. 3D is a figure illustrating the fourth step of the schematic process of producing a heart model by the rapid prototyping technique.

FIG. 4 is a figure illustrating a general schematic shape of a heart model on which surface a coronary artery is fixed.

FIG. 5 is a figure illustrating a mold (mold shell) for producing the coronary artery illustrated in FIG. 4.

FIG. 6 is a figure illustrating an overall shape (whole shape) of a coronary artery produced by the mold (mold shell) illustrated in FIG. 5.

### DESCRIPTION OF EMBODIMENTS

A method of producing an organ model according to the present invention is specifically described referring to the attached drawings. In the method of producing an organ model described below, a heart is referred to as a human organ. Therefore, the organ model produced in the embodiment below is a heart model.

In the method of producing an organ model according to the present invention, firstly, a mold (mold shell) for producing an organ model is made using rapid prototyping. The mold forms a stereo model as a copy of a human organ itself having a hollow portion, a protruding wall, or the like therein. Therefore, a mold for producing an organ model according to the present invention is different from a trimming die for producing a typical industrial product in that the mold is destroyed after an injection molding material (material for forming an organ model) is filled into and cured. That is, a mold is produced for each organ model to be produced, and is not reusable.

A rapid prototyping apparatus is used for producing the mold as described above. The rapid prototyping apparatus irradiates curing light (ultraviolet light from an ultraviolet lamp in the embodiment) to the photocurable mold resin and the photocurable support resin which supports the mold resin so as to cure the mold resin with the support from the support resin. In the rapid prototyping apparatus, different types of photocurable resin (the mold resin and the support resin) are continuously layered on an object forming portion (work stage) with given layer thicknesses respectively. The ultraviolet light from the ultraviolet lamp is irradiated to each gradually layered photocurable resin to obtain the desired stereo shape. In this case, the support resin contributes to supporting the mold resin and forming the stereo shape (outer-shape body) and is finally removed from the obtained outer-shape body. Therefore, a material which can easily be removed from the mold resin is used as the support resin. For example, a low melting-point resin having a low melting-point compared to the mold resin, or a water-soluble resin which easily dissolves in water can be used. In the embodiment, a photocurable acrylic resin having high water resistance is used as the mold resin, and a water-soluble resin which can easily be removed from the mold resin which is to be used is used as the support resin. The mold (mold shell) is produced by using a photocuring type 3D-printer (e.g., AGILISTA-3000 manufactured by Keyence Corp.) in which such mold resin and support resin can be used,

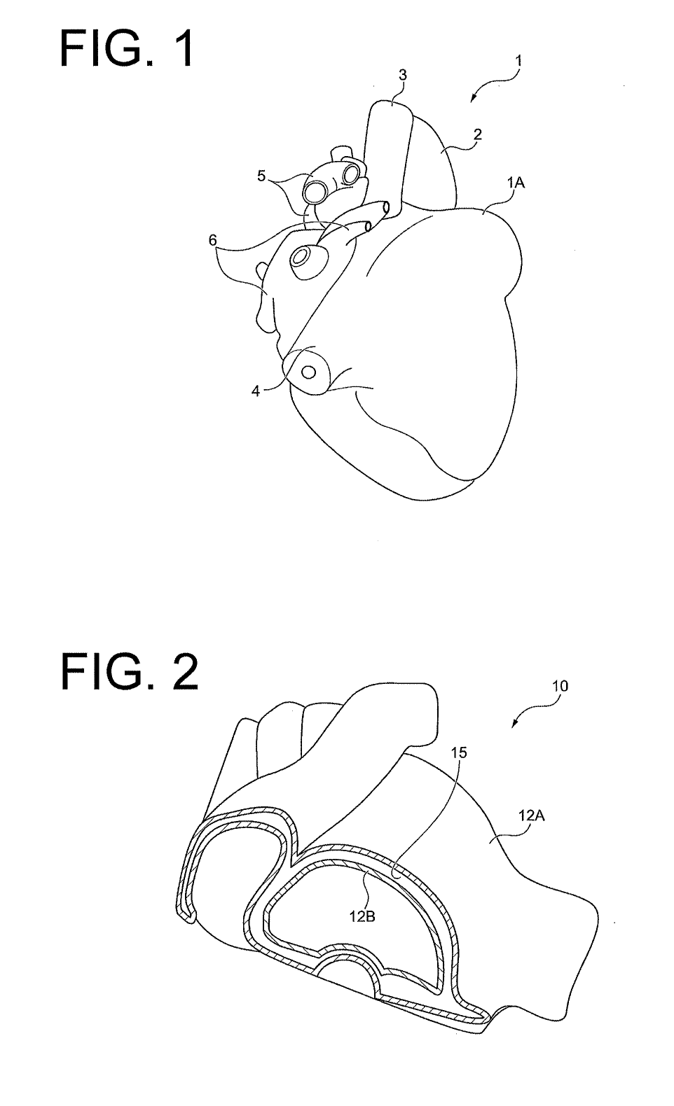

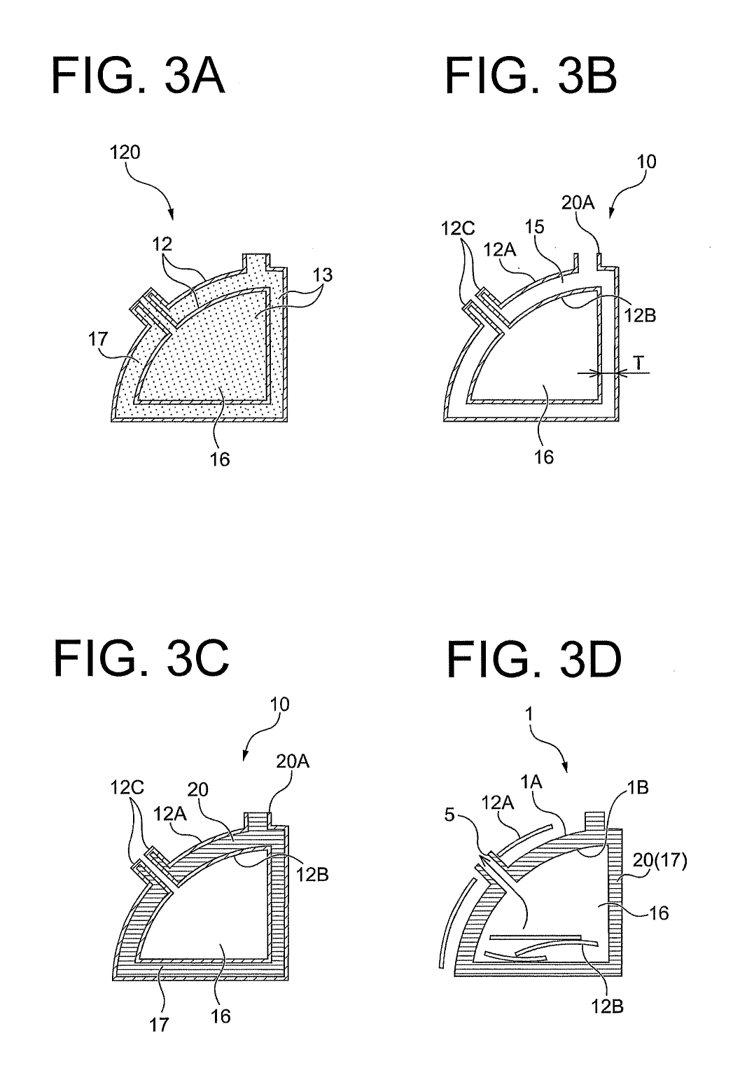

As illustrated in FIG. 1, the heart model 1 of the embodiment is a precise copy of an actual human heart (not illustrated in the drawing) and the overall shape is determined by the outer surface (outer layer portion) 1A. The heart model 1 has, similar to the actual heart, a region to be a hollow potion, specifically, a ventricle portion (a left ventricle and a right ventricle) and an atrium portion (a left atrium and a right atrium) therein. Further, on the surface portion of the heart model 1, a composing tissue which is connected to the ventricle portion and the atrium portion such as a main artery 2, a superior vena cava 3, an inferior vena cava 4, a pulmonary artery 5, and a pulmonary vein 6 is formed. In FIG. 1, though not illustrated in the drawing, an inner surface (underside layer portion) determining the ventricle portion and the atrium portion is appended with the numeral 1B (see FIG. 3A to FIG. 3D described below).

FIG. 2 illustrates a mold (mold shell) 10 for producing the heart model 1 illustrated in FIG. 1. As for the mold shell 10 illustrated in the drawing, the right atrium side is cut so as that the structure of the mold shell 10 can easily be understood. The mold shell 10 has an outer shell portion 12A which covers the outer surface 1A of the heart model 1 and an inner shell portion 12B which covers the inner surface 1B of the heart model 1. The heart model illustrated in FIG. 1 is produced by filling the space 15 between the outer shell portion 12A and the inner shell portion 12B of the mold shell 10 illustrated in FIG. 2 with the flexible injection molding material, and removing the mold shell 10 after the filled injection molding material is cured.

The process of producing the heart model 1 illustrated in FIG. 1 will specifically be described referring to FIG. 3A to FIG. 3D. Since the shape of the actual heart is complicated, the shape of the heart in FIG. 3A to 3D is illustrated in a simple schematic form, for ease of understanding.

First, the photographed data of the heart, for example, 2-dimensional tomographic image data, is obtained. As generally known, the 2-dimensional tomographic image data (hereinafter referred to as tomographic image data) is obtained by photographing an actual human body by an image photographing apparatus represented by a CT scanner. From the tomographic image data, the outer surface shape and the inner surface shape of the heart can be determined. The inside of the inner surface will be the internal space (hollow portion) which determines the ventricle, the atrium, or the like. The thick portion between the inner surface and the outer surface will be a so-called structural wall portion which determines the shape of the actual heart.

Then, by using the rapid prototyping apparatus, a photocurable mold resin 12 and a photocurable support resin 13 which supports the mold resin are continuously layered on an object forming portion (work stage) with given layer thicknesses respectively. The ultraviolet light from the ultraviolet lamp is irradiated to each of the gradually layered mold resin 12 and the support resin 13 based on the obtained tomographic image data of the heart to form the desired stereo shape corresponding to the heart. In this case, the ultraviolet light is irradiated to each of the photocurable resins 12 and 13 so that the support resin 13 is cured while supporting the mold resin 12 which is also cured. Thereby, as illustrated in FIG. 3A, an outer-shape body 120 having a region 16 which will finally be the hollow portion and a region 17 which will finally be the structural wall of the heart model is formed.

Then, the support resin 13 is removed from the outer-shape body 120. As mentioned above, the support resin 13 is composed of a water-soluble resin material, and thus can easily be removed by immersing in water (rinse water) so that the support resin absorbs moisture and suctions water to dissolve by itself. In this case, a plurality of holes is preferably provided on the outer side of the outer-shape body 120 so that the area in which the rinse water contacts the support resin 13 can be improved, thereby raising washing efficiency. Such hole having a diameter of about 1 mm is enough, and can be provided when the outer-shape body 120 is formed or after the outer-shape body 120 is formed. After the support resin 13 is removed, the hole is sealed by an adhesive or the like.

Further, by adding an alcohol or a surfactant to the rinse water, the solubility to the support resin 13 increases, and thereby the support resin 13 can efficiently be removed from the mold resin 12. Instead of the above-mentioned technique, other techniques may be employed such as agitating the rinse water by a magnet stirrer, a water pump, or the like, raising the temperature of the rinse water by a heater or the like, washing by micro-nano bubbles, ultrasonic cleaning, and pressurizing to a high pressure by a pressure chamber. Further, by suitably combining these techniques, the support resin 13 can efficiently and completely be removed from the mold resin 12.

As mentioned above, when the support resin 13 is removed, the outer-shape body 120 becomes a mold shell 10 as illustrated in FIG. 3B. The mold shell 10 includes the outer shell portion 12A which covers the outer surface 1A of the heart model 1 illustrated in FIG. 1 and the inner shell portion 12B which covers the inner surface 1B of the heart model 1. The thickness T of the space 15 between the outer shell portion 12A and the inner shell portion 12B of the mold shell 10 corresponds to the thickness of the heart model (thickness of the structural wall, assumed to be about 2 mm to 10 mm). The space 15 is filled with a flexible injection molding material 20.

The inner shell portion 12B is supported by the outer shell portion 12A via the space 15 (held in air or floating). As a support member which supports the inner shell portion 12B in position, a component of the heart model such as an ascending aorta, a superior vena cava, and/or an inferior vena cava can be used. These are open portions protruding outside from the hollow portion 16 inside the heart model and the edge of the opening becomes the support portion 12C which supports the inner shell portion 12B. Thus, during the rapid prototyping, the inner shell portion 12B is supported against the outer shell portion 12A. Further, a filling port 20A for filling the middle material 20 is formed on a portion of the outer shell portion 12A during rapid prototyping. In this case, a plurality of filling ports 20A may be formed so as that the injection molding material 20 is uniformly distributed throughout the space 15.

As illustrated in FIG. 3C, the space 15 between the outer shell portion 12A and the inner shell portion 12B of the mold shell 10 produced as mentioned above, is filled with the flexible injection molding material 20 via the filling port 20A. In this case, before filling with the injection molding material 20, a mold lubricant (release agent) or a coating material is preferably applied to the region of the mold shell 10 facing the space 15 so that the mold is easily removed and copying of roughness of the surface is prevented. Further, since the injection molding material 20 finally becomes a material composing the heart model, a material having hardness, texture, or the like similar to those of the actual heart is used. For example, a polymer gel material such as silicone (addition type/condensation type), urethane, and PVA (polyvinyl alcohol) can be used.

Further, as the filling material, a material which finally becomes transparent or can optionally be colored after curing may preferably be used. That is, by using a transparent type of material, the behavior during a treatment such as operating of the catheter or positioning of a stent (the insertion passage of the catheter or the location and state of the positioned stent) can visually be checked, thereby allowing an efficient simulation. Further, in the case of a material with colored appearance, the situation precisely close to an actual treatment can be replayed, thereby allowing a practical simulation.

In the embodiment, an additional type silicone having an excellent characteristic of transparency and flexibility is used. In this case, thinner may preferably be mixed in the injection molding material to improve handleability during injection molding. By mixing the thinner, the viscosity of the injection molding material is reduced, thereby easing the operation of injection molding.

Since a characteristic such as strength decreases when the thinner is mixed too much, the thinner is preferably mixed by 10 to 50 wt %. Further, since the space 15 of the above-mentioned mold shell 10 is sealed and the highly transparent silicone is used, a decompression-defoaming process may preferably be carried out at the time of filling so as to remove foam. That is, by carrying out the decompression-defoaming process when the injection molding material is filled, foam which is likely to remain in a corner region is removed and a heart model having extremely higher transparency can be obtained.

Further, as illustrated in FIG. 3D, after the filled injection molding material 20 is cured, the mold is removed by destroying (removing) the mold shell 10. In this case, a highly water-resistance photocurable acrylic resin (mold resin 12) is used for the mold shell 10. This material has low heat resistance and chemical resistance so that the material can easily be removed using the method described below.

The above-mentioned mold resin 12 softens at the temperature over about 50° C., and thereby can be removed by applying the softening temperature so as to cause deformation (splitting). In this case, the inner shell portion 12B can be removed from the open portion 5 of the ascending aorta, the superior vena cava, or the like which is a component of the heart. Alternatively, by immersing the mold resin in an organic solvent such as acetone to cause softening or crazing (cracking on the surface), the mold resin can be deformed so as to be removed easily, similar to the case of softening by heat. Further, after the mold resin 12 is crazed, by lowering the temperature of the mold resin 12 below the normal temperature, the mold resin 12 becomes fragile and can be destroyed more easily. By creating a fine crazing on the inner shell portion 12B so as to turn the inner shell portion 12B into fine particles, the damage on the copied structure (heart model) can be prevented, and also by the stream of air or the like, the destroyed inner shell portion can easily be removed from the internal space of the heart model.

Further, by coating the surface of the heart model 1 formed of the transparent material with the same type of material, the copied roughness can be filled so that the transparency can further be improved.

According to the above-mentioned method of producing the organ model, even for an organ model of an organ having a complicated internal shape as illustrated in FIG. 1, such as a heart, the organ model (heart model) is produced by forming the mold shell 10 using the mold resin 12 and the support resin 13, and finally destroying the mold shell 10. Therefore, compared to a conventional method of producing an organ model using an outer mold and a core, the direction to remove the mold or the location of the split are not necessary to be considered in the present invention, which makes it easier to produce a precise model. Further, the forming of the mold shell 10 does not include a plurality of copying processes, so that the production process is easier, thereby reducing cost and enabling the production of an organ model with high accuracy. Still further, in the case of copying an organ model unique to a patient, the cost of a mold can be reduced since the mold shell 10 itself is structurally disposable.

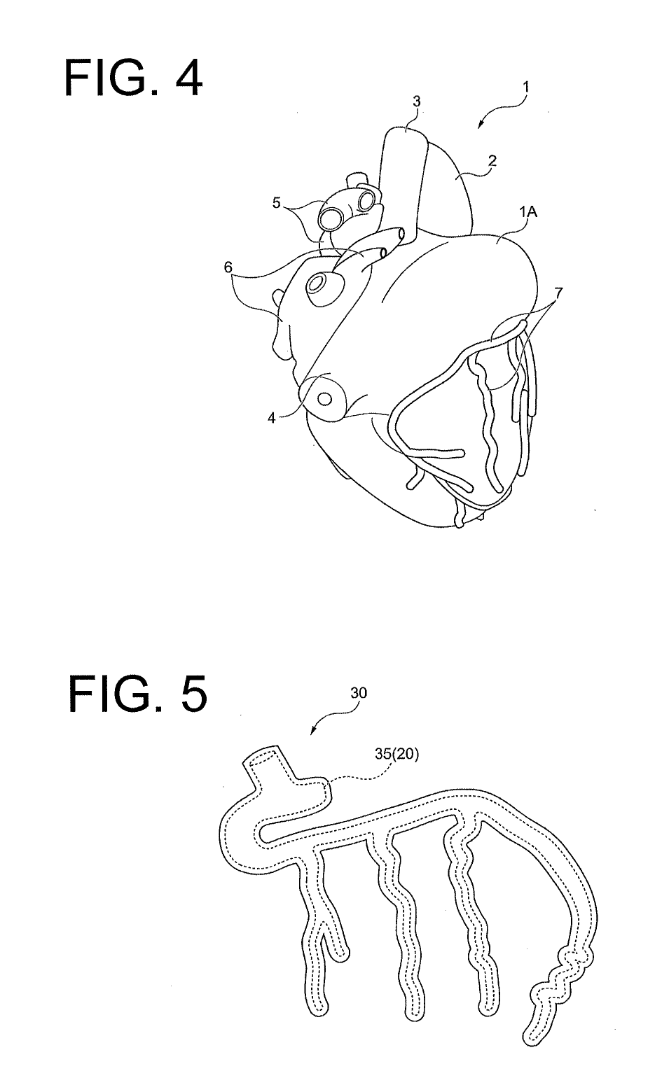

In the actual human heart, other than the above-mentioned ascending aorta, superior vena cava, and inferior vena cava, a coronary artery(s) which supplies blood exists on the surface of the main body of the heart. By the above-mentioned method, it is difficult to precisely copy the coronary artery 7 complicatedly arranged on the surface of the main body of the heart as illustrated in FIG. 4.

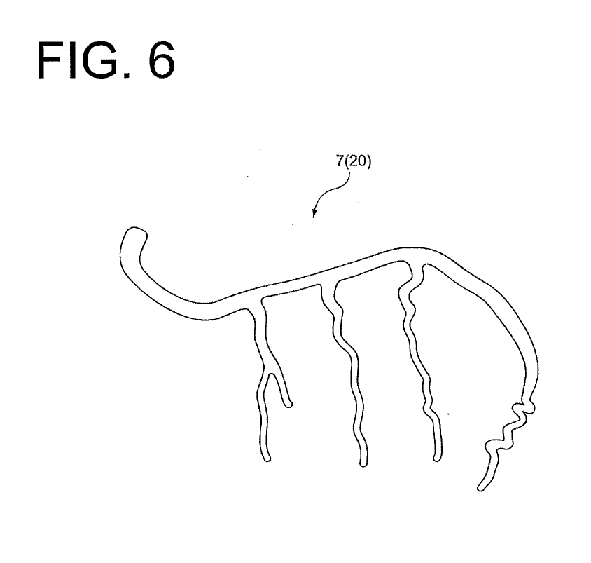

Therefore, as illustrated in FIG. 5, it is preferable to independently produce the coronary artery by the technique similar to the method of producing a heart model as mentioned above. Specifically, by using such mold resin and such support resin as mentioned above, a tubular mold shell 30 is formed, and the space 35 is filled with an injection molding material 20 similar to that of the above-mentioned embodiment. After the injection molding material 20 is cured, the mold shell 30 is removed, and thereby the coronary artery 7 as illustrated in FIG. 6 can independently be produced. By fixing the coronary artery 7 produced in such manner on the surface of the heart model 1 obtained by the method described above by an adhesive, a heart model further close to the actual heart can be produced.

The embodiment of the present invention is described above. However, the present invention is not limited to the configuration of the above-mentioned embodiment, and various modifications can be made.

In the above-mentioned embodiment, an explanation is made using a heart as an example. However, the present invention can be applied to a human organ other than the heart in a similar manner. Further, composing materials of the mold resin and the support resin, the injection molding material, and the method for removing the support material and the method of removing the mold shell may suitably be modified according to an organ to be produced or an application.

### REFERENCE SIGNS LIST

1: Heart model
10: Mold shell
12: Mold resin
12A: Outer shell portion
12B: Inner shell portion
13: Support resin
15: Space
16: Region to be hollow portion
17: Region to be structural wall
20: Injection molding material
120: Outer-shape body

1. A method of producing an organ model having a hollow portion inside thereof comprising:
an outer-shape body forming step in which an outer-shape body having a region which is to be the hollow portion and a region which is to be a structural wall of the organ model is formed by irradiating curing light to a photocurable mold resin and a photocurable support resin which supports the mold resin so as to cure the mold resin with support from the support resin based on photographed data of a human organ;
a mold shell forming step in which a mold shell having an outer shell portion which covers an outer surface of the organ model and an inner shell portion which covers an inner surface of the organ model is formed by removing the support resin from the outer-shape body;
a filling step in which a space between the outer shell portion and the inner shell portion of the mold shell is filled with a flexible injection molding material; and
a removing step in which the mold shell is removed after the injection molding material is filled.

2. The method of producing an organ model according to claim 1, wherein the support resin is a water-soluble resin and the support resin is removed by washing the outer-shape body with a rinse water to form the mold shell.

3. The method of producing an organ model according to claim 2, wherein a plurality of holes are formed on the outer-shape body.

4. The method of producing an organ model according to claim 2, wherein an alcohol or a surfactant is added to the rinse water.

5. The method of producing an organ model according to claim 1, wherein the injection molding material is a material having transparency.

6. The method of producing an organ model according to claim 5, wherein a decompression-defoaming process is carried out to remove foam when filling the injection molding material.

7. The method of producing an organ model according to claim 5, wherein a surface of a human organ formed of the material having transparency is coated with a similar type of material.

8. An organ model produced by the method of producing an organ model according to claim 1.

9. A mold for producing an organ model, for producing an organ model having a hollow portion inside thereof, wherein
an outer-shape body having a region which is to be the hollow portion and a region which is to be a structural wall of the organ model is formed by irradiating a photocurable mold resin and a photocurable support resin which supports the mold resin with curing light so as to cure the mold resin with support from the support resin based on photographed data of a human organ; and
an outer shell portion which covers an outer surface of the organ model and an inner shell portion which covers an inner surface of the organ model are formed by removing the support resin from the outer-shape body.
